(19) United States
(12) Patent Application Publication
(10) Pub. No.: US 2025/0121165 A1
(43) Pub. Date: Apr. 17, 2025

(54) Guidewire-Management Devices and Methods Thereof

(71) Applicant: Bard Access Systems, Inc., Salt Lake City, UT (US)

(72) Inventor: Glade H. Howell, Draper, UT (US)

(21) Appl. No.: 19/000,076

(22) Filed: Dec. 23, 2024

Related U.S. Application Data
(62) Division of application No. 17/079,320, filed on Oct. 23, 2020, now Pat. No. 12,186,503.
(60) Provisional application No. 62/926,327, filed on Oct. 25, 2019.

(51) Int. Cl. A61M 25/09; A61M 25/00; A61M 25/01

(52) U.S. Cl. CPC A61M 25/09041; A61M 25/0136; A61M 2025/0019

(57) ABSTRACT

Guidewire-management devices including a guidewire, a first sleeve, and a second sleeve. The first sleeve can be configured for distally feeding the guidewire out of the guidewire-management device. The first sleeve can also be configured for proximally feeding the guidewire into the guidewire-management device. The second sleeve can be proximal of the first sleeve in the guidewire-management device. The second sleeve can be configured for feeding the guidewire in concert with the first sleeve. At least a length of the guidewire extending between the first sleeve and the second sleeve can be disposed within a sterile barrier configured to maintain sterility of the guidewire.

### CROSS-REFERENCE

This application is a division of U.S. patent application Ser. No. 17/079,320, filed Oct. 23, 2020, now U.S. Pat. No. 12,186,503, which claims the benefit of priority to U.S. Provisional Application No. 62/926,327, filed Oct. 25, 2019, each of which is incorporated by reference in its entirety into this application.

### BACKGROUND

A catheter is typically placed in a blood vessel of a patient using the Seldinger technique, which involves inserting a hollow needle into the blood vessel, inserting a guidewire into the needle, advancing the guidewire into the blood vessel, and removing the needle from the blood vessel leaving the guidewire in place. The catheter is then advanced over the guidewire until it is suitably placed with the patient's vasculature. The guidewire is then removed from the patient.

Guidewires for use in accordance with the Seldinger technique are typically packaged in a coiled-tube dispenser to keep the guidewires sterile and make handling the guidewires less cumbersome. However, inserting such a guidewire from a dispenser into a hollow needle requires two hands. In particular, one hand is required to hold both the needle and an engagement piece configured to guide the guidewire into the needle, while another hand is required to grip the guidewire and direct the guidewire from the dispenser into the needle. The more the guidewire is handled, the more opportunity for contamination or damage to the guidewire. In view of the foregoing, there is a need for better guidewire management in medical procedures such as those requiring the Seldinger technique.

Disclosed herein are guidewire-management devices and methods thereof that address the foregoing.

### SUMMARY

Disclosed herein is a guidewire-management device including, in some embodiments, a guidewire, a first sleeve, and a second sleeve. The first sleeve is configured for distally feeding the guidewire out of the guidewire-management device. The first sleeve is also configured for proximally feeding the guidewire into the guidewire-management device. The second sleeve is proximal of the first sleeve in the guidewire-management device. The second sleeve is configured for feeding the guidewire in concert with the first sleeve. At least a length of the guidewire extending between the first sleeve and the second sleeve is disposed within a sterile barrier configured to maintain sterility of the guidewire.

In some embodiments, the first sleeve has a distal-end portion configured as a male connector with a Luer taper for connecting the first sleeve to a complementary female connector.

In some embodiments, the first sleeve is configured to straighten the guidewire when proximally feeding the guidewire into the guidewire-management device.

In some embodiments, the first sleeve includes a seal configured to block fluid from entering or escaping the guidewire-management device when proximally feeding the guidewire into the guidewire-management device.

In some embodiments, the first sleeve has a proximal-end portion including a bore configured to house a distal-end portion of the sterile barrier.

In some embodiments, the second sleeve has a distal-end portion including a bore configured to house a proximal-end portion of the sterile barrier.

In some embodiments, the second sleeve has a proximal-end portion including another bore configured to stop an end of the guidewire from completely passing through the second sleeve.

In some embodiments, the guidewire has a distal-end portion including a ‘J’-shaped tip configured to straighten as the tip of the guidewire enters the first sleeve when proximally feeding the guidewire into the guidewire-management device.

In some embodiments, the guidewire has a proximal-end portion including a ball end configured to stop the guidewire from completely passing through the second sleeve.

In some embodiments, the sterile barrier is a pleated bag. The bag is configured to unpleat as it is drawn from the second sleeve while distally feeding the guidewire out of the guidewire-management device. The bag is also configured to pleat as it is inserted into the first sleeve while distally feeding the guidewire out of the guidewire-management device.

In some embodiments, the sterile barrier is a bellowed boot configured to stretch from an equilibrium state and relax back into the equilibrium state while distally feeding the guidewire out of the guidewire-management device.

Also disclosed herein is a guidewire-management device including, in some embodiments, a guidewire, a frame including a pair of tubular rails, a guidewire conduit threaded through the tubular rails, a first sleeve mounted between each rail of the pair of rails, and a second sleeve mounted between each rail of the pair of rails proximal of the first sleeve. The guidewire includes a distal-end portion including a ‘J’-shaped tip and a proximal-end portion including a ball end. The guidewire is disposed within the guidewire conduit. The first sleeve is configured for distally feeding the guidewire out of the guidewire-management device. The first sleeve is also configured for proximally feeding the guidewire into the guidewire-management device. The second sleeve is configured for feeding the guidewire in concert with the first sleeve. At least a length of the guidewire extending between the first sleeve and the second sleeve is disposed within a sterile barrier configured to maintain sterility of the guidewire.

In some embodiments, the first sleeve has a distal-end portion configured as a male connector with a Luer taper for connecting the first sleeve to a complementary female connector.

In some embodiments, the first sleeve is configured to straighten the tip of the guidewire as the tip of the guidewire enters the first sleeve when proximally feeding the guidewire into the guidewire-management device.

In some embodiments, the second sleeve has a proximal-end portion including a bore with a constriction configured to capture the ball end of the guidewire and stop the guidewire from completely passing through the second sleeve.

In some embodiments, the guidewire conduit has a proximal-end portion including a plug configured to stop the ball end of the guidewire from passing through an opening in a proximal end of the guidewire conduit. Stopping the ball end of the guidewire from passing through the opening in the guidewire conduit prevents the tip of the guidewire from completely passing through the first sleeve and reforming the ‘J’-shaped tip when proximally feeding the guidewire into the guidewire-management device.

In some embodiments, the sterile barrier is a pleated bag. The bag is configured to unpleat as it is drawn from a bore in a distal-end portion of the second sleeve while distally feeding the guidewire out of the guidewire-management device. The bag is also configured to pleat as it is inserted into a bore in a proximal-end portion of the first sleeve while distally feeding the guidewire out of the guidewire-management device.

In some embodiments, the sterile barrier is a bellowed boot configured to stretch from an equilibrium state and relax back into the equilibrium state while distally feeding the guidewire out of the guidewire-management device.

Also disclosed herein is a guidewire-management device including, in some embodiments, a handle, a guidewire, a first sleeve formed in a distal-end portion of the handle, a second sleeve formed in a proximal-end portion of the handle, and a thumb wheel disposed under the guidewire between the first sleeve and the second sleeve. At least a length of the guidewire extending between the first sleeve and the second sleeve is disposed within a sterile barrier configured to maintain sterility of the guidewire. The thumb wheel is configured to assist in distally feeding the guidewire out of the guidewire-management device by way of the first sleeve. The thumb wheel is also configured to assist in proximally feeding the guidewire into the guidewire-management device by way of the first sleeve.

In some embodiments, the first sleeve has a distal-end portion configured as a male connector with a Luer taper for connecting the first sleeve to a complementary female connector.

In some embodiments, the first sleeve is configured to straighten a ‘J’-shaped tip in a distal-end portion of the guidewire as the tip of the guidewire enters the first sleeve when proximally feeding the guidewire into the guidewire-management device.

In some embodiments, the second sleeve has a proximal-end portion including a bore with a constriction configured to capture a ball end of the guidewire and stop the guidewire from completely passing through the second sleeve.

In some embodiments, the sterile barrier is a pleated bag over an entirety of the guidewire excepting that within or distal to the first sleeve. The bag is configured to pleat as the bag is inserted into a bore in a proximal-end portion of the first sleeve while distally feeding the guidewire out of the guidewire-management device.

In some embodiments, a proximal end of the bag is closed providing a proximal limit to proximally feeding the guidewire into the guidewire-management device by way of the first sleeve. The proximal limit established by the proximal end of the bag is configured to prevent the ‘J’-shaped tip of the guidewire from passing completely through the first sleeve when proximally feeding the guidewire into the guidewire-management device by way of the first sleeve.

Also disclosed herein is method of a guidewire-management device including, in some embodiments, a connecting step of connecting a first sleeve of the guidewire-management device to a hub of a medical device inserted into an insertion site of a patient. The first sleeve is configured as a male connector with a Luer taper. The method further includes an advancing step of advancing a guidewire of the guidewire-management device from the first sleeve through the hub of the medical device and into the insertion site of the patient. The guidewire is disposed within a sterile barrier configured to maintain sterility of the guidewire until being fed into the first sleeve.

In some embodiments, the advancing step includes pinching the guidewire within the sterile barrier and advancing the guidewire into the first sleeve by hand. The sterile barrier is a pleated bag or a bellowed boot.

In some embodiments, the advancing step includes pressing the guidewire within the sterile barrier onto a thumb wheel and rolling the thumb wheel to advance the guidewire into the first sleeve, the sterile barrier being a pleated bag.

In some embodiments, the method further includes a ceasing step of ceasing to advance the guidewire into the insertion site of the patient when a ball end of a proximal-end portion of the guidewire is captured in a constriction of a bore in a proximal-end portion of a second sleeve of the guidewire-management device.

In some embodiments, the method further includes withdrawing the guidewire from the insertion site of the patient through the hub of the medical device and into the first sleeve. A seal in the first sleeve blocks fluid from entering or escaping the guidewire-management device when withdrawing the guidewire from the insertion site.

In some embodiments, a distal-end portion of the guidewire includes a ‘J’-shaped tip configured to straighten as the tip of the guidewire enters the first sleeve when withdrawing the guidewire from the insertion site.

In some embodiments, the method further includes ceasing to withdraw the guidewire from the insertion site of the patient when a proximal end of the guidewire abuts a plug in a proximal-end portion of a guidewire conduit of the guidewire-management device.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a first guidewire-management device in accordance with some embodiments.

FIG. 2 illustrates a longitudinal cross section of the first guidewire-management device in accordance with some embodiments.

FIG. 3 illustrates a second guidewire-management device in accordance with some embodiments.

FIG. 4 illustrates a longitudinal cross section of the second guidewire-management device in accordance with some embodiments.

FIG. 5 illustrates a ball end of a guidewire distal to a plug in a guidewire conduit in accordance with some embodiments.

FIG. 6 illustrates a third guidewire-management device in accordance with some embodiments.

FIG. 7 illustrates a detailed view of a handle of the third guidewire-management device in accordance with some embodiments.

FIG. 8 illustrates a longitudinal cross section of the third guidewire-management device in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

Unless defined otherwise, all technical and scientific terms used herein have the same meaning as commonly understood by those of ordinary skill in the art.

As set forth above, there is a need for better guidewire management in medical procedures such as those requiring the Seldinger technique. Disclosed herein are guidewire-management devices and methods thereof that address the foregoing.

### Guidewire-Management Devices

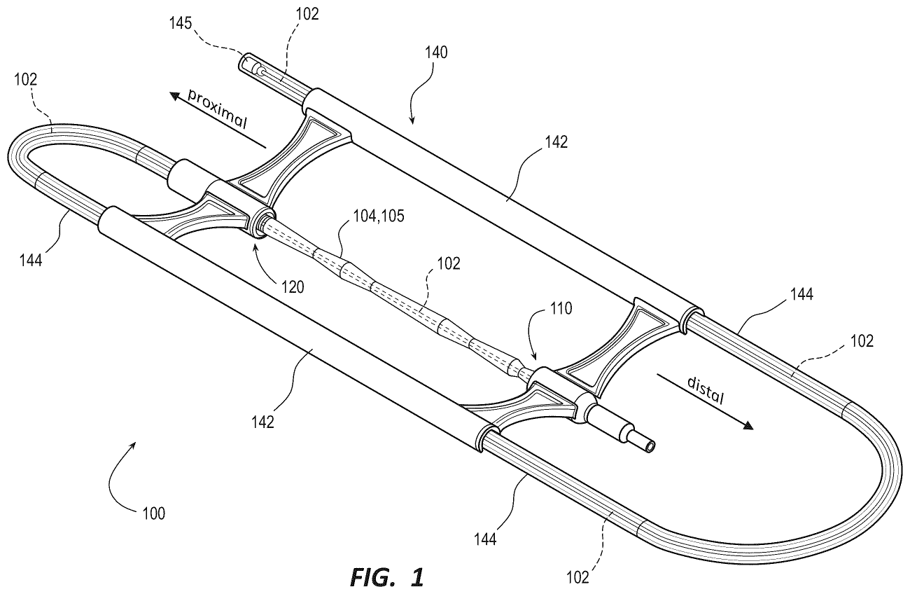

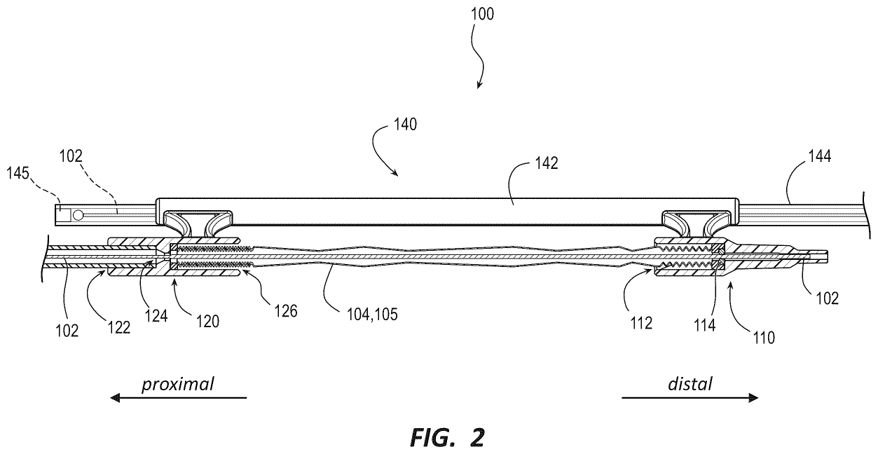

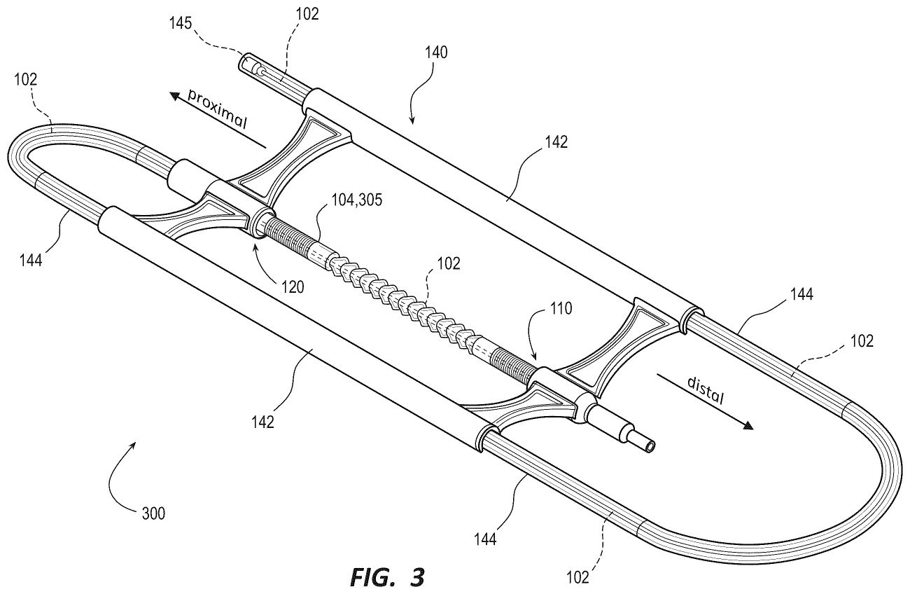

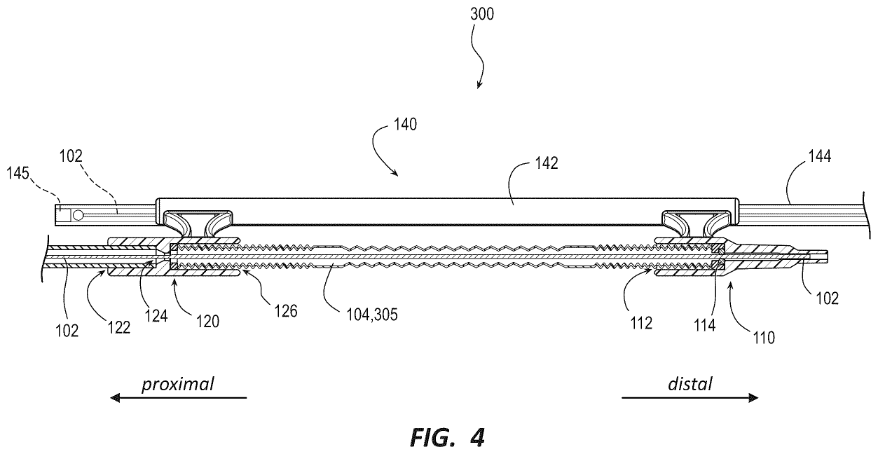

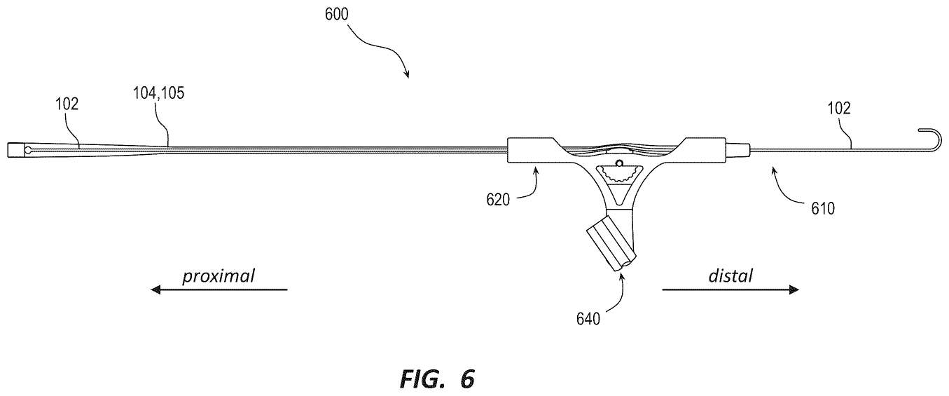

FIGS. 1 and 2 illustrate a first guidewire-management device 100 in accordance with some embodiments. FIGS. 3 and 4 illustrate a second guidewire-management device 300 in accordance with some embodiments. FIGS. 6-8 illustrates a third guidewire-management device 600 in accordance with some embodiments. The guidewire-management devices 100, 300, and 600 are initially described below with an emphasis on their common features. Subsequently, the guidewire-management devices 100, 300, and 600 are described below primarily with respect to their unique features.

As shown, the guidewire-management device 100, 300, or 600 includes a guidewire 102, a first sleeve 110 or 610, and a second sleeve 120 or 620.

The first sleeve 110 or 610 is configured for distally feeding, or advancing, the guidewire 102 out of the guidewire-management device 100, 300, or 600. The first sleeve 110 or 610 has a distal-end portion configured as a male connector with a Luer taper for connecting the first sleeve 110 or 610 to a complementary female connector such as that of a hub of a hollow needle, which facilitates one-handed operation of the guidewire-management device 100, 300, or 600 including distally feeding the guidewire 102 out of the guidewire-management device 100, 300, or 600. The first sleeve 110 or 610 also has a proximal-end portion including a bore 112 or 712 configured to house a distal-end portion of a sterile barrier 104 or collect the distal-end portion of the sterile barrier 104 when distally feeding the guidewire 102 out of the guidewire-management device 100, 300, or 600.

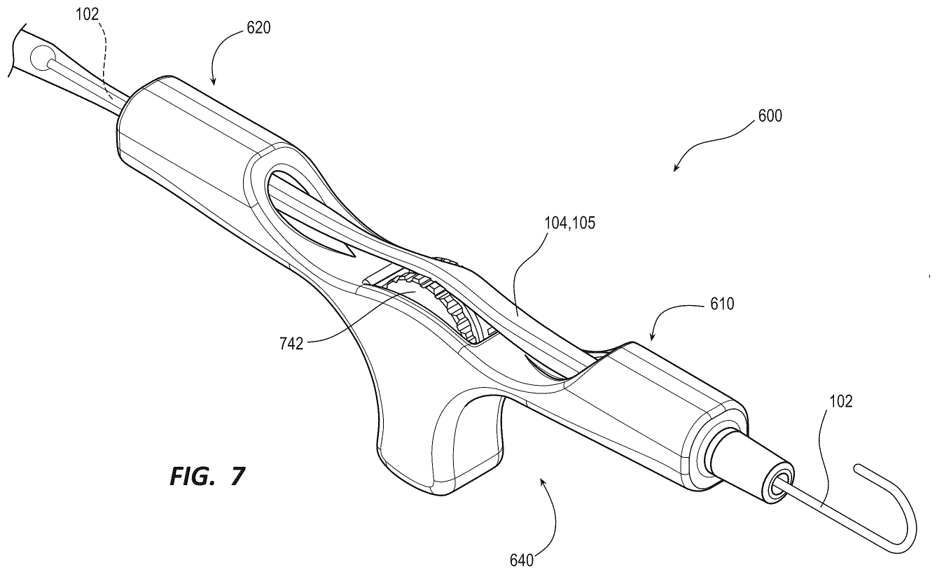

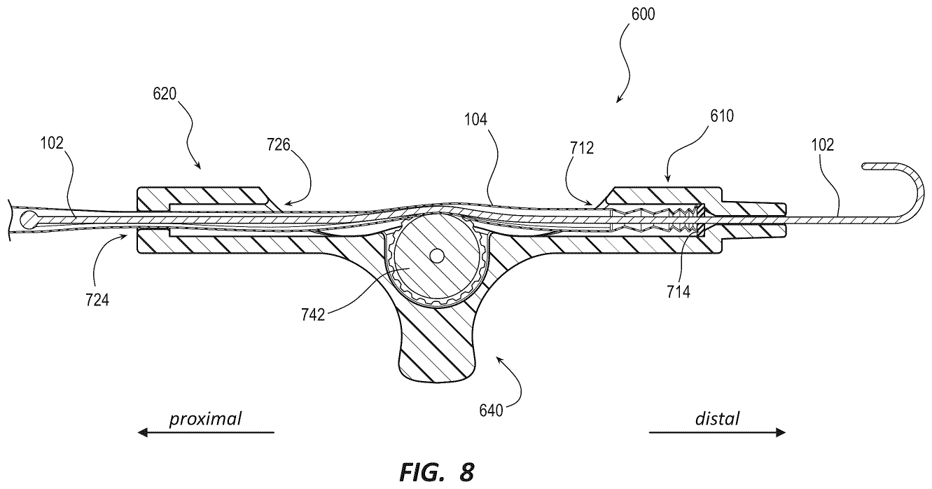

The first sleeve 110 or 610 is also configured for proximally feeding, or withdrawing, the guidewire 102 into the guidewire-management device 100, 300, or 600. The first sleeve 110 or 610 is configured to straighten the guidewire 102 such as a ‘J’-shaped tip thereof when proximally feeding the guidewire 102 into the guidewire-management device 100, 300, or 600. (See FIGS. 7 and 8 for the ‘J’-shaped tip of the guidewire 102 in accordance with some embodiments.) The first sleeve 110 or 610 can include a seal such as an ‘O’-ring 114 or 714 configured to block fluid (e.g., blood) from entering the guidewire-management device 100, 300, or 600 when proximally feeding the guidewire into the guidewire-management device 100, 300, or 600. The seal, for example, the ‘O’-ring 114 or 714, is also configured to block fluid (e.g., air) from escaping the guidewire-management device 100, 300, or 600 when proximally feeding the guidewire into the guidewire-management device 100, 300, or 600, thereby minimizing or obviating risk of air embolism.

The second sleeve 120 or 620 is proximal of the first sleeve 110 or 610 in the guidewire-management device 100, 300, or 600 such that at least a length of the guidewire 102 extends between the first sleeve 110 or 610 and the second sleeve 120 or 620. At least the length of the guidewire 102 extending between the first sleeve 110 or 610 and the second sleeve 120 or 620 is disposed within the sterile barrier 104.

The second sleeve 120 or 620 is configured for feeding the guidewire 102 in concert with the first sleeve 110 or 610 such as distally feeding the guidewire 102 out of the guidewire-management device 100, 300, or 600. The second sleeve 120 or 620 has a proximal-end portion optionally including a bore 122. For example, the second sleeve 120 has the proximal-end portion including the bore 122, whereas the second sleeve 620 has the proximal-end portion without such a bore. Whether or not the second sleeve 120 or 620 includes such a bore, the second sleeve 120 or 620 further includes a constriction 124, a recess 724, or a similar stopping means for stopping an end of the guidewire 102 such as a ball end thereof from completely passing through the second sleeve 120 or 620 when distally feeding the guidewire 102 out of the guidewire-management device 100, 300, or 600. Since the constriction 124, the recess 724, or the similar stopping means is configured to stop the end of the guidewire 102 from completely passing through the second sleeve 120 or 620, the constriction 124, the recess 724, or the similar stopping means is also configured to stop the guidewire from completely passing through the guidewire-management device 100, 300, or 600 and into a patient where the guidewire 102 can be lost.

The second sleeve 120 or 620 is also configured for proximally feeding the guidewire 102 into of the guidewire-management device 100, 300, or 600 in concert with the first sleeve 110 or 610. The second sleeve 120 or 620 has a distal-end portion including a bore 126 or 726 configured to house a proximal-end portion of the sterile barrier 104 or collect the proximal-end portion of the sterile barrier 104 when proximally feeding the guidewire 102 into of the guidewire-management device 100, 300, or 600.

The guidewire 102 has a distal-end portion and a proximal-end portion.

The distal-end portion of the guidewire 102 can include a ‘J’-shaped tip configured to straighten as the tip of the guidewire 102 enters the first sleeve 110 or 610 when proximally feeding the guidewire 102 into the guidewire-management device 100, 300, or 600. When distally feeding the guidewire 102 out of the guidewire-management device 100, 300, or 600, the ‘J’-shaped tip is configured to reform the ‘J’ shape upon release from its constriction (e.g., the first sleeve 110 or 610). If the first sleeve 110 or 610 is connected to a complementary female connector such as that of a hub of a hollow needle, the tip of the guidewire 102 does not reform the ‘J’ shape until the tip is distally beyond the needle such as in a blood vessel.

The proximal-end portion of the guidewire 102 can include a ball end configured to stop the guidewire 102 from completely passing through the second sleeve 120 or 620 on account of the constriction 124, the recess 724, or the similar stopping means when distally feeding the guidewire 102 out the guidewire-management device 100, 300, or 600.

The sterile barrier 104 is configured to maintain sterility of the guidewire 102 while the guidewire 102 is disposed therein. Importantly, the guidewire never needs to be touched by a clinician during a procedure, thereby preserving the sterility of the guidewire 102 as well as ensuring the clinician remains free from contact with bodily fluids.

The sterile barrier 104 can be a pleated bag 105. The bag 105 is configured to unpleat as it is drawn from the second sleeve 120 or 620 while distally feeding the guidewire 102 out of the guidewire-management device 100 or 600. The bag 105 is also configured to pleat as it is inserted into the first sleeve 110 or 610 while distally feeding the guidewire 102 out of the guidewire-management device 100 or 600. Likewise, the bag 105 is configured to unpleat as it is drawn from the first sleeve 110 or 610 while proximally feeding the guidewire 102 into the guidewire-management device 100 or 600. The bag 105 is also configured to pleat as it is inserted into the second sleeve 120 or 620 while proximally feeding the guidewire 102 into the guidewire-management device 100 or 600.

With respect to at least the guidewire-management device 600, a proximal end of the bag 105 is closed and, optionally, coupled to, for example, the ball end of the guidewire 102, which provides a proximal limit to proximally feeding the guidewire 102 into the guidewire-management device 600 by way of the first sleeve 610. The proximal limit, which is established by the proximal end of the bag 105 when the bag 105 is fully extended, is configured to prevent the ‘J’-shaped tip of the guidewire 102 from passing completely through the first sleeve 610 when proximally feeding the guidewire 102 into the guidewire-management device 600 by way of the first sleeve 610. This is advantageous for it would be particularly difficult to reinsert the ‘J’-shaped tip of the guidewire 102 back into the first sleeve 610 if the ‘J’-shaped tip of the guidewire 102 passes completely through the first sleeve 610 when proximally feeding the guidewire 102 into the guidewire-management device 600.

A method for distally feeding, or advancing, the guidewire 102 out of the guidewire-management device 100 or 600 is set forth below. A method for proximally feeding, or withdrawing, the guidewire 102 into the guidewire-management device 100 or 600 is also set forth below.

The sterile barrier 104 can be a bellowed boot 305. The boot 305 is configured to iteratively stretch from an equilibrium state and relax back into the equilibrium state while distally feeding the guidewire 102 out of the guidewire-management device 300. Likewise, the boot 305 is configured to iteratively stretch from an equilibrium state and relax back into the equilibrium state while proximally feeding the guidewire 102 into the guidewire-management device 600.

A method for distally feeding, or advancing, the guidewire 102 out of the guidewire-management device 300 is set forth below. A method for proximally feeding, or withdrawing, the guidewire 102 into the guidewire-management device 300 is also set forth below.

Adverting to FIGS. 1-4 to describe some of the features unique to the guidewire-management devices 100 and 300, the guidewire-management devices 100 and 300 include the guidewire 102, a frame 140 including a pair of tubular rails 142, a guidewire conduit 144 threaded through the pair of rails 142, the first sleeve 110 mounted on struts between each rail of the pair of rails 142, and the second sleeve 120 mounted on struts between each rail of the pair of rails 142 proximal of the first sleeve 110. The guidewire 102 is disposed within the guidewire conduit 144.

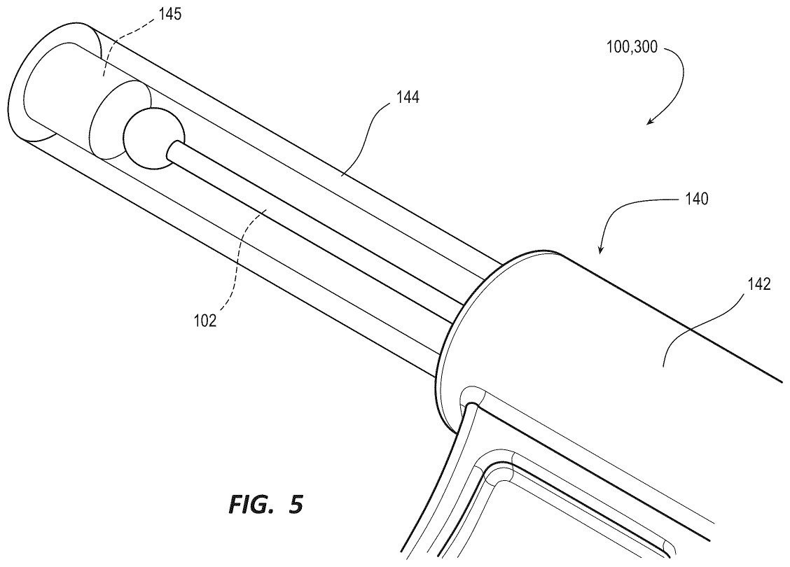

FIG. 5 illustrates the ball end of the guidewire 102 distal to a plug 145 in the guidewire conduit 144 in accordance with some embodiments.

The guidewire conduit 144 has a proximal-end portion including the plug 145 configured to stop the ball end of the guidewire 102 from passing through what would otherwise be an opening in a proximal end of the guidewire conduit 144. Stopping the ball end of the guidewire 102 from passing through the opening in the guidewire conduit 144 prevents the tip of the guidewire 102 from completely passing through the first sleeve 110 and reforming the ‘J’-shaped tip when proximally feeding the guidewire 102 into the guidewire-management device 100 or 300.

Adverting to FIGS. 6-8 to describe some of the features unique to the guidewire-management device 600, the guidewire-management device 600 includes a handle 640, the guidewire 102, the first sleeve 610 formed in a distal-end portion of the handle 640, the second sleeve 620 formed in a proximal-end portion of the handle 640, and a thumb wheel 742 disposed under the guidewire 102 between the first sleeve 610 and the second sleeve 620.

The thumb wheel 742 is mounted on an axle connected to each side of the handle 640. The thumb wheel 742 is configured to assist in distally feeding the guidewire 102 out of the guidewire-management device 600 by way of the first sleeve 610. The thumb wheel 742 is also configured to assist in proximally feeding the guidewire 102 into the guidewire-management device 600 by way of the first sleeve 610.

A method for distally feeding, or advancing, the guidewire 102 out of the guidewire-management device 600 is set forth below. A method for proximally feeding, or withdrawing, the guidewire 102 into the guidewire-management device 600 is also set forth below.

### Methods

A method of the guidewire-management device 100, 300, or 600 includes a connecting step of connecting the first sleeve 110 or 610 of the guidewire-management device 100, 300, or 600 to a hub of a medical device (e.g., a hollow needle) inserted into an insertion site of a patient.

The method further includes an advancing step of advancing the guidewire 102 of the guidewire-management device 100, 300, or 600 from the first sleeve 110 or 610 through the hub of the medical device and into the insertion site of the patient. The seal (e.g., the ‘O’-ring 114 or 714) in the first sleeve 110 or 610 blocks fluid (e.g., air) from escaping the guidewire-management device 100, 300, or 600 when advancing the guidewire 102 through the hub of the medical device and into the insertion site of the patient.

The advancing step can include pinching the guidewire 102 within the sterile barrier 104 and advancing the guidewire 102 into the first sleeve 110 by hand. The advancing can alternatively include pressing the guidewire 102 within the sterile barrier 104 onto the thumb wheel 742 and rolling the thumb wheel 742 to advance the guidewire 102 into the first sleeve 610.

Indeed, the guidewire 102 can be advanced into the first sleeve 110 or 610 and out of the guidewire-management device 100 or 600 by pinching a combination of the guidewire 102 within the bag 105 or pressing the guidewire 102 within the bag 105 against the thumb wheel 742, advancing the guidewire 102 into the first sleeve 110 by hand or by rolling the thumb wheel 742, releasing the combination of the guidewire 102 within the bag 105, and repeating the foregoing as necessary. The guidewire 102 can be advanced into the first sleeve 110 and out of the guidewire-management device 300 by pinching a combination of the guidewire 102 within the boot 305, advancing the guidewire 102 into the first sleeve 110 by hand, releasing the combination of the guidewire 102 within the boot 305, and repeating the foregoing as necessary.

The method can further include a ceasing step of ceasing to advance the guidewire 102 into the insertion site of the patient when the ball end of the guidewire 102 is captured in the constriction 124 of the bore 122 in the second sleeve 120 of the guidewire-management device 100 or 300. Likewise, the ceasing step can include ceasing to advance the guidewire 102 into the insertion site of the patient when the ball end of the guidewire 102 is captured in the recess 724 in the second sleeve 620 of the guidewire-management device 600.

The method further includes withdrawing the guidewire 102 from the insertion site of the patient through the hub of the medical device and into the first sleeve 110 or 610. The seal (e.g., the ‘O’-ring 114 or 714) in the first sleeve 110 or 610 blocks fluid (e.g., blood) from entering the guidewire-management device 100, 300, or 600 when withdrawing the guidewire 102 from the insertion site.

Indeed, the guidewire 102 can be withdrawn into the guidewire-management device 100 or 600 through the first sleeve 110 or 610 by pinching the combination of the guidewire 102 within the bag 105 or pressing the guidewire 102 within the bag 105 against the thumb wheel 742, withdrawing the guidewire 102 from the first sleeve 110 by hand or by rolling the thumb wheel 742, releasing the combination of the guidewire 102 within the bag 105, and repeating the foregoing as necessary. The guidewire 102 can be withdrawn into the guidewire-management device 300 through the first sleeve 110 by pinching the combination of the guidewire 102 within the boot 305, withdrawing the guidewire 102 from the first sleeve 110 by hand, releasing the combination of the guidewire 102 within the boot 305, and repeating the foregoing as necessary.

The method can further include ceasing to withdraw the guidewire 102 from the insertion site of the patient when the proximal end or the ball end of the guidewire 102 abuts the plug 145 in the proximal-end portion of the guidewire conduit 144 of the guidewire-management device 100 or 300.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A guidewire-management device, comprising:
a handle;
a guidewire including a ‘J’-shaped tip in a distal-end portion of the guidewire;
a first sleeve formed in a distal-end portion of the handle;
a second sleeve formed in a proximal-end portion of the handle, wherein a length of the guidewire extends between the first sleeve and the second sleeve within a sterile barrier configured to maintain sterility of the guidewire; and
a thumb wheel disposed under the guidewire between the first sleeve and the second sleeve configured to assist in feeding the guidewire distally out of the guidewire-management device or proximally into the guidewire-management device.

2. The guidewire-management device according to claim 1, wherein the first sleeve has a distal-end portion configured as a male connector with a Luer taper for connecting the first sleeve to a complementary female connector.

3. The guidewire-management device according to claim 1, wherein the first sleeve is configured to straighten a ‘J’-shaped tip in a distal-end portion of the guidewire as the tip of the guidewire enters the first sleeve when proximally feeding the guidewire into the guidewire-management device.

4. The guidewire-management device according to claim 1, wherein the guidewire includes a ball on a proximal end, and wherein the second sleeve has a proximal-end portion including a bore with a constriction configured to capture the ball and stop the guidewire from moving further distally.

5. The guidewire-management device according to claim 1, wherein the sterile barrier is a pleated bag over an entirety of the guidewire excepting that within or distal to the first sleeve configured to pleat as the bag is inserted into a bore in a proximal-end portion of the first sleeve while distally feeding the guidewire out of the guidewire-management device.

6. The guidewire-management device according to claim 1, wherein the sterile barrier is a bellowed boot configured to stretch from an equilibrium state and relax back into the equilibrium state while distally feeding the guidewire out of the guidewire-management device.

7. The guidewire-management device according to claim 1, wherein a proximal end of the bag is closed providing a proximal limit to proximally feeding the guidewire into the guidewire-management device by way of the first sleeve, the proximal limit configured to prevent the ‘J’-shaped tip of the guidewire from passing completely through the first sleeve when proximally feeding the guidewire into the guidewire-management device.

8. The guidewire-management device according to claim 1, wherein the first sleeve comprises a seal configured to block fluid from entering or escaping the guidewire-management device when proximally feeding the guidewire into the guidewire-management device.

9. The guidewire-management device according to claim 8, wherein a distal end of the sterile barrier is coupled to the seal.

10. A method of using a guidewire-management device, comprising:
grasping a handle of the guidewire-management device, the handle comprising a first sleeve formed in a distal-end portion of the handle and a second sleeve formed in a proximal-end portion of the handle, wherein a length of a guidewire of the guidewire-management device extends between the first sleeve and the second sleeve within a sterile barrier configured to maintain sterility of the guidewire;
connecting the first sleeve of the guidewire-management device to a hub of a medical device inserted into an insertion site of a patient; and
advancing the guidewire from the first sleeve through the hub of the medical device and into the insertion site of the patient by rotating a thumb wheel disposed under the guidewire between the first sleeve and the second sleeve.

11. The method according to claim 10, wherein advancing the guidewire includes pinching the guidewire within the sterile barrier and advancing the guidewire into the first sleeve by hand, the sterile barrier being a pleated bag or a bellowed boot.

12. The method according to claim 10, wherein advancing the guidewire includes pressing the guidewire within the sterile barrier onto the thumb wheel while rotating the thumb wheel to advance the guidewire into the first sleeve.

13. The method according to claim 10, further comprising ceasing to advance the guidewire into the insertion site of the patient when a ball end of a proximal-end portion of the guidewire is captured in a constriction of a bore in a proximal-end portion of the second sleeve of the guidewire-management device.

14. The method according to claim 10, further comprising withdrawing the guidewire from the insertion site of the patient through the hub of the medical device and into the first sleeve, a seal in the first sleeve blocking fluid from entering or escaping the guidewire-management device when withdrawing the guidewire from the insertion site.

15. The method according to claim 14, wherein a distal-end portion of the guidewire includes a ‘J’-shaped tip configured to straighten as the tip of the guidewire enters the first sleeve when withdrawing the guidewire from the insertion site.

16. The method according to claim 14, further comprising ceasing to withdraw the guidewire from the insertion site of the patient when a proximal end of the guidewire abuts a plug in a proximal-end portion of a guidewire conduit of the guidewire-management device.
